(19) United States
(12) Patent Application Publication
(10) Pub. No.: US 2012/0041068 A1
(43) Pub. Date: Feb. 16, 2012

(54) Extended Release Pharmaceutical Preparations for Active Pharmaceutical Ingredients with pH Dependent Solubility

(75) Inventor: Siva Rama Krishna Nutalapati, Princeton, NJ (US)

(73) Assignee: Aptapharma, Inc., Pennsauken, NJ (US)

(21) Appl. No.: 13/207,538

(22) Filed: Aug. 11, 2011

Related U.S. Application Data
(60) Provisional application No. 61/372,542, filed on Aug. 11, 2010.

(51) Int. Cl. A61K 31/165; A61P 25/28; A61P 9/12; A61P 25/22

(52) U.S. Cl. 514/617

(57) ABSTRACT

An extended release preparation of an active pharmaceutical ingredient with pH dependent solubility dissolved or dispersed in a semi-solid or liquid nonionic solubilizer and further containing a non-pH dependent sustained release agent as well as a process for the preparation thereof are provided.

This patent application claims the benefit of priority from U.S. Provisional Application Ser. No. 61/372,542, filed Aug. 11, 2010, the teachings of which are hereby incorporated by reference in their entirety.

### BACKGROUND OF THE INVENTION

The rate at which an active pharmaceutical ingredient goes into solution when dissolved in a medium is proportional to the solubility of the active pharmaceutical ingredient in the medium. Many active pharmaceutical ingredients have different solubilities at different pHs. These pH-dependent solubility differences lead to pH-dependent dissolution profiles. Tablets containing, for example, a basic drug can exhibit a faster dissolution profile in simulated gastric fluid, having a pH about 1.0, than in simulated intestinal fluid (pH 6.8 to 7.4).

In general, pH-dependent dissolution is an undesirable product characteristic.

U.S. Pat. No. 6,287,599 discloses pharmaceutical compositions having a pH-independent or a minimized pH-dependent dissolution profile. The composition includes at least one pharmaceutically active agent that has a pH dependent solubility profile, at least one non-pH-dependent sustained release agent, and at least one pH-dependent agent that increases the dissolution rate of the at least one pharmaceutically active agent at a pH in excess of 5.5.

### SUMMARY OF THE INVENTION

An aspect of the present invention relates to an extended release pharmaceutical composition comprising at least one active pharmaceutical ingredient that is pH dependent, at least one non-pH dependent sustained release agent, and at least one nonionic solubilizer.

In one embodiment, the composition further comprises a crystallization inhibitor. In one embodiment, the active pharmaceutical ingredient comprises guanfacine hydrochloride.

Another aspect of the present invention relates to a method for preparation of an extended release pharmaceutical composition comprising at least one active pharmaceutical ingredient that is pH dependent, wherein said active pharmaceutical ingredient is mixed with at least one nonionic solubilizer and at least one non-pH dependent sustained release agent and compressed into a tablet. In one embodiment, the active ingredient is mixed with said active pharmaceutical ingredient is mixed with at least one nonionic solubilizer, at least one non-pH dependent sustained release agent and a crystallization inhibitor and compressed into a tablet.

### DETAILED DESCRIPTION OF THE INVENTION

The present invention is related to pharmaceutical extended release preparations of at least one active pharmaceutical ingredient that is pH dependent and to methods for production of such preparations. The extended release preparations of the present invention show prolonged and nearly constant rate of absorption of the active pharmaceutical ingredient with pH dependent solubility for a long period of time and concurrently maintain a high extent of bioavailability. The preparations are achieved by using a nonionic solubilizer with or without solvent which is mixed with the active pharmaceutical ingredient with pH dependent solubility. The preparations further comprise a crystallization inhibitor and at least one non-pH dependent sustained release agent.

In one embodiment, the active pharmaceutical ingredient with pH dependent solubility is dissolved or dispersed in the nonionic solubilizer with the aid of a solvent. The mixture of active pharmaceutical ingredient and nonionic solubilizer with or without solvent can be further diluted with water or intestinal juice. In the solution the active pharmaceutical ingredient is included in a micell-structure formed by the solubilizer. The mixture of the active pharmaceutical ingredient and the solubilizer with or without solvent is incorporated into a pharmaceutical formulation, further comprising a crystallization inhibitor and at least one non-pH dependent sustained release agent which provide prolonged release.

In one embodiment, the active pharmaceutical ingredient has a solubility profile wherein the active agent is more soluble in an acidic medium than in a basic medium.

Active pharmaceutical ingredients with pH dependent solubility which may be included in the pharmaceutical extended release preparations of the present invention include, but are not limited to, weakly basic drugs and their salts that have higher solubilities at lower pH levels. Examples of such drugs include, but are not limited to, guanfacine hydrochloride, guanadrel sulfate, reserpine, anagrelide hydrochloride, propanolol, metoprolol, atenolol, timolol, erythromycin, clonidine, chlorpheniramine, bromopheniramine, diltiazen, and scopolamine. In one embodiment, the at least one active pharmaceutical ingredient is guanfacine hydrochloride. As the skilled artisan will understand upon reading this disclosure, however, the scope of the present invention is not limited to any particular pH dependent active pharmaceutical ingredient agent.

The active pharmaceutical ingredient is typically present in the composition in an amount of from about 0.1 wt. % to about 70 wt. %. In one embodiment the active pharmaceutical ingredient is present in the composition in an amount of from about 0.5 wt. % to about 40 wt %.

Nonionic solubilizers useful in the present invention are semi-solid or liquid nonionic surface active agents. In one embodiment, the solubilizer contains polyethylene glycols as esters or ethers. In one embodiment, the solubilizer is selected from polyethoxylated fatty acids, hydroxylated fatty acids and fatty alcohols. In one embodiment, the solubilizer is selected from polyethoxylated castor oil, polyethoxylated hydrogenated castor oil, polyethoxylated fatty acid from castor oil or polyethoxylated fatty acid from hydrogenated castor oil. Commercially available nonionic surface active agents which can be used are known under the trade names Cremophor, Myrj, Polyoxyl 40 stearate, Emerest 2675, Lipal 395, Tween, Span and HCO 50.

Nonionic solubilizers other than surface active agents useful in the present invention are nonvolatile liquids. Preferably these nonionic solubilizers are water miscible liquids. Examples of suitable nonvolatile solubilizers include, but are not limited to, liquid polyethylene glycols, propylene glycol, glycerin, N,N-dimethylacetamide.

Solvents useful in the present invention are volatile or non volatile liquids. Preferably these liquids are volatile organic solvents. Suitable volatile solvents include, but are not limited to, ethyl alcohol, denatured alcohol, ester, acetone, transcutol.

To prevent or minimize recrystallization of the active pharmaceutical ingredients and to be able to administer the therapeutically desired dose continuously, a crystallization inhibitor can be added (see Table 1). By addition of a crystallization inhibitor, a high portion of active pharmaceutical ingredient remains dissolved during the storage time. Crystallization inhibitors are therefore particularly useful in rendering the active pharmaceutical ingredient continuously bioavailable in humans at therapeutically relevant doses. For example, guanfacine in the presence of a crystallization inhibitor such as polyvinyl pyrrolidone exhibited less tendency toward crystal formation than a comparison formulation without polyvinyl pyrrolidone. As crystallization inhibitors, highly dispersed silicon dioxide or macromolecular substances are suitable. Examples of macromolecular substances include, but are not limited to, polyvinylpyrrolidones with an average molecular weight of about 1,000 to 2,000,000 (for example, Plasdone, Kollidon), vinylpyrrolidone-vinyl acetate copolymers (Kollidon VA 64), crosslinked polyvinylpyrrolidones (Kollidon CL), polyvinyl alcohol, hydroxypropyl cellulose, ethyl cellulose, gelatin, starch (derivatives), dextrins and dextrans, such as, for example, α-, β- and γ-cyclodextrin, dimethyl-βcyclodextrin and 2-hydroxypropyl-β-cyclodextrin), sterols (such as cholesterol) or bile acids (such as cholic acid or lithocholic acid).

TABLE 1
Effect of Crystallization inhibitor on guanfacine
HCl solubility

| | | Crystallization | Day | Day | Day |
|---|---|---|---|---|---|
| S. No | Drug:Chremophor | Inhibitor | 0 | 5 | 20 |
| 1 | 1_2 | None | Clear | Ppt | Ppt |
| 2 | 1_3 | None | Clear | Ppt | Ppt |
| 3 | 1_5 | None | Clear | Ppt | Ppt |
| 4 | 1_7 | None | Clear | Clear | Ppt |
| 5 | 1_10 | None | Clear | Clear | Clear |
| 6 | 1_2 | Polyvinyl pyrrolidone | Clear | Ppt | Ppt |
| 7 | 1_3 | Polyvinyl pyrrolidone | Clear | Ppt | Ppt |
| 8 | 1_5 | Polyvinyl pyrrolidone | Clear | Clear | Clear |
| 9 | 1_7 | Polyvinyl pyrrolidone | Clear | Clear | Clear |
| 10 | 1_10 | Polyvinyl pyrrolidone | Clear | Clear | Clear |
| 11 | 1_2 | Hydroxypropyl cellulose | Clear | Ppt | Ppt |
| 12 | 1_3 | Hydroxypropyl cellulose | Clear | Ppt | Ppt |
| 13 | 1_5 | Hydroxypropyl cellulose | Clear | Ppt | Ppt |
| 14 | 1_7 | Hydroxypropyl cellulose | Clear | Clear | Clear |
| 15 | 1_10 | Hydroxypropyl cellulose | Clear | Clear | Clear |

As shown in Table 1, polyyinylpyrrolidone exhibited high crystallization-inhibitory potency.

Non-pH-dependent sustained release agents which can be used in the present invention include, but are not limited to, ethylcellulose, cellulose acetate, vinyl acetate/vinyl chloride copolymers, acrylate/methacrylate copolymers, polyethylene oxide, hydroxypropyl methylcellulose, carrageenan, alginic acid and salts thereof, hydroxyethyl cellulose, hydroxypropyl cellulose, karaya gum, acacia gum, tragacanth gum, locust bean gum, guar gum, sodium carboxymethyl cellulose, methyl cellulose, beeswax, carnauba wax, cetyl alcohol, hydrogenated vegetable oils, and stearyl alcohol. As will be understood by the skilled artisan upon reading this disclosure, however, the scope of the present invention is not to be limited to any particular non-pH-dependent sustained release agent.

The at least one non-pH-dependent sustained release agent is typically present in the composition in an amount of from about 5 wt. % to about 50 wt. %. In one embodiment, the at least one non-pH-dependent sustained release agent is present in the composition in an amount of from about 5 wt. % to about 30 wt. %.

The active pharmaceutical ingredient mixed with the solubilizer with or without the aid of a solvent and preferably with a crystallization inhibitor can be incorporated into different kinds of known controlled release systems such as, but not limited to a hydrophilic gel system, an erosion system, beads coated with a rate controlling membrane which can be a diffusion retarding coating or a disintegrating coating, and tablets with an inert porous matrix.

In one embodiment, the solubilized active pharmaceutical ingredient is combined with a hydrophilic gel system, namely a hydrophilic swelling matrix such as, but not limited to, hydroxypropyl methyl cellulose. This form of controlled release mechanism is a suitable way to control the release of the micelles of drug and solubilizer. Other examples of suitable compounds effecting the release of the active compound from the hydrophilic gel system are guar gum, xanthan gum, carboxypolymethylene, different cellulosic materials e.g. sodium carboxymethylcellulose and hydroxypropyl cellulose, lactose and aluminium silicate.

The preparation according to this embodiment of the invention contains 10-80% by weight, preferably 15-50% by weight of the hydrophilic gel system.

The final preparation in this embodiment is, for example, in the form of a gel tablet. By a careful choice of fillers and binders as well as gel forming material the preparation can be manufactured into a commercially acceptable form, e.g. a tablet or a hard gelatin capsule comprising the gel forming granulate, that shows unexpectedly good absorption of the active compound as well as a prolonged duration of action. In the preparation according to the invention the proportions between the active compound and the solubilizer varies in the range from 1:1 to 1:20, preferably in the range from 1:2 to 1:10 and similarly the solvent varies in the range from 1:1 to 1:20, preferably in the range from 1:2 to 1:10.

Other types of controlled release preparations which can be used in accordance with the present invention include tablets with an inert porous matrix; capsules comprising granules with a diffusion retarding coating or a disintegrating coating.

In one embodiment; tablets with an inert porous matrix are obtained by mixing the active pharmaceutical ingredient and the solubilizer with or without the aid of a solvent and preferably a crystallization inhibitor with water insoluble polymers or waxes and with fillers and binders. Polyvinylacetate, polyvinylchloride, ethylcellulose, paraffin and cellulose acetate phthalate could be used as suitable diffusion-retarding polymers. The fillers and binders are solid, powdered carriers such as lactose, saccharose, sorbitol, mannitol, starch, amylopectin, cellulose derivative, gelatine or other suitable carrier. Also a lubricating agent, such as, but not limited to, magnesium stearate, calcium stearate, sodium stearyl fumarate and polyethyleneglycol wax may be added. The mixture is then formed into tablets.

In one embodiment, capsules comprising granules with extended release characteristics are obtained by making a core material consisting of the active pharmaceutical ingredient, solubilizer_and preferably a crystallization inhibitor together with fillers. The surface of the core is then coated with diffusion-retarding water insoluble pH independent polymers or waxes. The granules are then filled into hard gelatin capsules. In one embodiment, the core material is prepared by mixing the active pharmaceutical ingredient, solubilizer and crystallization inhibitor, with or without solvent, and granulating the mixture with carefully selected fillers such as lactose, sorbitol, starch, cellulose derivatives or other suitable fillers. The mass is formed to granules by, for example, extrusion and spheronization. The surfaces of the cores formed are coated with a solution consisting of a solvent such as, but not limited to, methylene chloride and/or isopropyl alcohol and water insoluble pH independent polymers such as, but not limited to, ethylcellulose. The granules are filled in hard gelatine capsules.

Preparations of the present invention may further include other materials such as bulking agents, disintegrating agents, anti-adherants and glidants, lubricants, and binding agents.

Bulking agents include, but are not limited to, microcrystalline cellulose (e.g. Avicel, FMC Corp., Emcocel, Mendell Inc.), mannitol, xylitol, dicalcium phosphate (e.g. Emcompress, Mendell Inc.), calcium sulfate (e.g. Compactrol, Mendell Inc.), starches, lactose, sucrose (e.g. Dipac, Amstar, and Nutab, Ingredient Technology), dextrose (e.g. Emdex, Mendell, Inc.), sorbitol, and cellulose powder (e.g. Elcema, Degussa, and Solka Floc, Mendell, Inc.). The bulking agent may be present in the preparation in an amount of from about 5 wt. % to about 90 wt. %, preferably from about 10 wt. % to about 50 wt. %.

Disintegrating agents which may be included in the preparations include, but are not limited to, microcrystalline cellulose, starches, crospovidone (e.g. Polyplasdone XL, International Specialty Products.), sodium starch glycolate (e.g. Explotab, Mendell Inc.), and crosscarmellose sodium (e.g. Ac-Di-Sol, FMC Corp.). The disintegrating agent may be present in the preparation in an amount of from about 0.5 wt. % to about 30 wt %, preferably from about 1 wt. % to about 15 wt. %.

Antiadherants and glidants which may be employed in the preparation include, but are not limited to, talc, corn starch, silicon dioxide, sodium lauryl sulfate, and metallic stearates. The antiadherant or glidant may be present in the preparation in an amount of from about 0.2 wt. % to about 15 wt. %, preferably from about 0.5 wt. % to about 5 wt. %.

Lubricants which may be employed in the preparation include, but are not limited to, magnesium stearate, calcium stearate, sodium stearate, stearic acid, sodium stearyl fumarate, hydrogenated cotton seed oil (e.g. sterotex), talc, and waxes, including but not limited to, bees wax, carnuba wax, cetyl alcohol, glyceryl stearate, glyceryl palmitate, glyceryl behenate, hydrogenated vegetable oils, and stearyl alcohol. The lubricant may be present in an amount of from about 0.2 wt. % to about 20 wt. %, preferably from about 0.5 wt. % to about 5 wt. %.

Binding agents which may be employed include, but are not limited to, polyvinyl pyrrollidone, starch, methylcellulose, hydroxypropyl methylcellulose, carboxymethyl cellulose, sucrose solution, dextrose solution, acacia, tragacanth and locust bean gum. The binding agent may be present in the preparation in an amount of from about 0.2 wt. % to about 10 wt. %, preferably from about 0.5 wt. % to about 5 wt. %.

In one embodiment, preparations of the present invention are made by a wet granulation method.

In the wet granulation method, the at least one pharmaceutically active agent is either dissolved or dispersed in the solubilizer with or without a solvent. To prevent crystallization either during storage or in vivo use a crystallization inhibitor is added. This solution or dispersion is used to granulate the other ingredients. If necessary, additional amount of solvent can be added to improve the granulation. The wet granules are dried in an oven or fluidized bed dryer, and then sieved through a suitable screen to obtain free flowing granules. The resulting granules were blended with a suitable lubricant and glidant, and the lubricated granules are compressed into tablets on a rotary press using appropriate tooling. If desired, a coating can be applied onto the compressed tablets.

The following nonlimiting examples are provided to further illustrate the present invention.

### EXAMPLES

### Example 1

### Preparation of Guanfacine HCl Extended Release Tablets

| | Kg/batch |
|---|---|
| Guanfacine HCl | 0.912 |
| Cremophor RH40 | 0.912 |
| SD3A* | 2.000 |
| PVP K 90 | 1.000 |
| HPMC K100 MCR | 12.000 |
| PVP K 90 | 4.000 |
| Lactose Fast flo | 17.000 |
| Avicel PH200 | 10.176 |
| Syloid | 1.000 |
| Magnesium Stearate | 1.000 |
| | 48.000 |

Mix SD3A alcohol and Chremophor. Dissolve Guanfacine HCl in the above solution. Add PVP and mix. In a collette mix HPMC, PVP, Lactose and Avicel. Granulate the powder mix with the above drug solution. Dry the granules and size using a Fitz mill. Screen Syloid and Magnesium stearate separately. Blend the sized granules and screened materials in a V blender. Compress into tablets of 250 mg each.

### Example 2

### Preparation of Guanfacine HCl Extended Release Tablets

| | Kg/batch |
|---|---|
| Guanfacine HCl | 0.912 |
| Cremophor RH40 | 0.912 |
| SD3A* | 2.000 |
| PVP K 90 | 4.000 |
| Carnauba wax | 10.000 |
| Klucel HXF | 3.000 |
| Lactose Fast flo | 17.000 |
| Avicel PH200 | 10.176 |
| Syloid | 1.000 |
| Magnesium Stearate | 1.000 |
| | 48.000 |

Mix SD3A alcohol and Chremophor. Dissolve Guanfacine HCl in the above solution. Add PVP and mix. In a collette mix Carnauba wax, Klucel, Lactose and Avicel. Granulate the powder mix with the above drug solution. Dry the granules and size using a Fitz mill. Screen Syloid and Magnesium stearate separately. Blend the sized granules and screened materials in a V blender. Compress into tablets of 250 mg each.

What is claimed is:

1. A pharmaceutical composition comprising:
(a) at least one active pharmaceutical ingredient with pH dependent solubility;
(b) at least one non-pH dependent sustained release agent; and
(c) at least one nonionic solubilizer.

2. The pharmaceutical composition of claim 1 further comprising a crystallization inhibitor.

3. The pharmaceutical composition of claim 1 further comprising a solvent.

4. The pharmaceutical composition of claim 1 wherein the nonionic solubilizer is a semi-solid or liquid nonionic surface active agent.

5. The pharmaceutical composition of claim 4 wherein the nonionic solubilizer is selected from esters and/or ethers of polyethylene glycols.

6. The pharmaceutical composition of claim 4 wherein the nonionic solubilizer is selected from polyethoxylated fatty acids, hydroxylated fatty acids or fatty alcohols.

7. The pharmaceutical composition of claim 4 wherein the nonionic solubilizer is selected from polyethoxylated castor oil, polyethoxylated hydrogenated castor oil, a polyethoxylated fatty acid from castor oil or a polyethoxylated fatty acid from hydrogenated castor oil.

8. The pharmaceutical composition of claim 4 wherein the nonionic solubilizer is an ester of hydrogenated castor oil fatty acids with oxyethylated glycerine.

9. The pharmaceutical composition of claim 4 wherein the nonionic solubilizer is Cremophor, Myrj, Polyoxyl 40 stearate, Emerest 2675, Lipal 395, Tween, Span and HCO 50.

10. The pharmaceutical composition of claim 4 wherein the nonionic solubilizer is liquid polyethylene glycols, propylene glycol, glycerin, N,N-dimethylacetamide.

11. The pharmaceutical composition of claim 2 wherein the crystallization inhibitor is a polyvinylpyrrolidone, vinylpyrrolidone-vinyl acetate copolymer, polyvinyl alcohol, hydroxypropyl cellulose, ethyl cellulose, gelatin, starch (derivatives), dextrin, dextran, sterol or bile acid.

12. The pharmaceutical composition of claim 3 wherein the solvent is ethyl alcohol, denatured alcohol, ester, acetone or transcutol.

13. The pharmaceutical composition of claim 1 wherein the active pharmaceutical ingredient is selected from guanfacine hydrochloride, guanadrel sulfate, reserpine, anagrelide hydrochloride, propanolol, metoprolol, atenolol, timolol, erythromycin, clonidine, chlorpheniramine, bromopheniramine, diltiazen or scopolamine.

14. The pharmaceutical composition of claim 1 wherein the active pharmaceutical ingredient is guanfacine hydrochloride.
